(19) United States
(12) Patent Application Publication
(10) Pub. No.: US 2026/0027760 A1
(43) Pub. Date: Jan. 29, 2026

(54) Surface Texturing of Silicone Elastomer Using Polymeric Bags

(71) Applicant: American Breast Care, LP, Marietta, GA (US)

(72) Inventor: Amanda Wollnick, Doraville, GA (US)

(21) Appl. No.: 18/784,691

(22) Filed: Jul. 25, 2024

(51) Int. Cl. B29C 45/37; B29C 33/42; B29K 83/00; B29L 31/00

(52) U.S. Cl. CPC B29C 45/372; B29C 33/424; A61F 2/12; A61F 2/52; B29K 2083/005; B29K 2875/00; B29L 2031/7532

(57) ABSTRACT

In a method of applying a desired texture to a surface of a silicone elastomer, an uncured silicone elastomer is applied to at least one side of a polymeric film. At least one side of the polymeric film has a complementary texture that is complementary in shape to the desired texture. The uncured silicone elastomer is cured while it is in contact with the at least one side of the polymeric film so as to form a cured silicone elastomer. The polymeric film is removed from the cured silicone elastomer so as to expose the desired texture on the surface thereof. The flexible polymeric film can be a bag placed into a breast prosthesis mold. The bag includes at least one interior surface with the complementary texture. The bag is removed from the cured silicone elastomer to expose the breast prosthesis having a textured surface.

### BACKGROUND OF THE INVENTION

### 1. Field of the Invention

The present invention relates to textured silicone elastomers and, more specifically, to a method of making a textured silicone elastomer using a polymeric bag.

### 2. Description of the Related Art

Silicone elastomers can mirror the surface it is cast or molded against exactly and can mirror fine details, textures and even microtextures.

Having a silicone elastomer with a surface texture can be desirable for several reasons. Textured or microtextured surfaces can help reduce the shiny look of cured silicone elastomer and create a softer hand feel with reduced friction. Other benefits of texturing silicone elastomer may include reduced bacterial adhesion or enhanced biocompatibility of silicone implants and medical devices. The texture may also simply be decorative, such as adding patterns (stripes, dots, lace, etc.) or designs.

There are several methods to create textures on cured silicone elastomer surfaces, but existing methods can also have disadvantages when used in certain applications. A silicone elastomer mold may be carved, etched, sanded, or have a textured coating applied to the mold surface. In many molding applications, a mold release agent is needed to remove the cured silicone elastomer from the mold. The mold release agent can fill in the textures and impact the effect or look of the texture. Also, if a textured mold is used repeatedly to make molded silicone elastomer parts, the texture can be prone to damage or being worn away. Also, a mold will require frequent cleaning or re-application of the texture to the surface.

Another texturing process involves using dissolvable granules or powder, such as salt or sugar. The dissolvable granules or powder can be applied to the mold surface prior to application of uncured silicone. Alternatively, the silicone elastomer surface while still partially uncured and ‘wet’ can be either dipped into or dusted with the dissolvable granules or powder. After the silicone elastomer cures, the dissolvable granules or powder are removed with a solvent, leaving a textured surface. However, handling powders can be messy. Also, this process requires the extra steps of dissolving the granules or powder and then drying off any residual solvent. There is also possibility of the granules or powder becoming permanently embedded into the silicone elastomer surface, which may not be desirable.

Another texturing method involves embedding an open cell foam or mesh polymeric fabric into the uncured silicone elastomer surface. The foam is dissolved with harsh solvents to create a texture. These solvents may require special personal protective equipment and ventilation when handling. There is also an additional step of removing any residual solvent. As for the fabric, it is removed from the silicone elastomer surface before the silicone elastomer has fully cured. This method requires perfect timing. If the fabric is removed too soon, no surface texture will remain on the surface of the cured silicone elastomer. If the fabric is removed too late, it will then be embedded within the cured silicone elastomer. Additionally, when handling delicate, partially cured silicone elastomers, it is possible to distort the desired shape, smudge or damage the surface. Also, dust, dirt, or lint can fall onto the partially cured silicone elastomer surface during the multiple processing steps.

Therefore, there is a need for method of applying a texture to a surface of a silicone elastomer that reliably applies the desired texture to the silicone elastomer with minimal complexity.

### SUMMARY OF THE INVENTION

The disadvantages of the prior art are overcome by the present invention which, in one aspect, is a method of applying a desired texture to a surface of a silicone elastomer, in which uncured silicone elastomer is applied to at least one side of a polymeric film. At least one side of the polymeric film has a complementary texture that is complementary in shape to the desired texture. The uncured silicone elastomer is cured while it is in contact with the at least one side of the polymeric film so as to form a cured silicone elastomer. The polymeric film is removed from the cured silicone elastomer so as to expose the desired texture on the surface thereof.

In another aspect, the invention is a method of making a silicone elastomer breast prosthesis, in which a bag that includes a flexible polymeric film is placed into a mold that is complementary in shape to the breast prosthesis. The polymeric bag includes at least one interior surface that has a complementary texture that is complementary to a desired texture. An uncured silicone elastomer is injected into the bag. The uncured silicone elastomer is cured while the uncured silicone elastomer is in the bag so as to form a cured silicone elastomer shape. The bag is removed from the silicone elastomer shape so as to expose the breast prosthesis. The breast prosthesis includes at least one surface having the desired texture.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIGS. 1A-1E are a series of schematic diagrams demonstrating one method of applying a texture to a silicone elastomer breast prosthesis.

### DETAILED DESCRIPTION OF THE INVENTION

A preferred embodiment of the invention is now described in detail. Referring to the drawings, like numbers indicate like parts throughout the views. Unless otherwise specifically indicated in the disclosure that follows, the drawings are not necessarily drawn to scale. The present disclosure should in no way be limited to the exemplary implementations and techniques illustrated in the drawings and described below. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

One embodiment uses a textured polymer film applied to a surface of a silicone elastomer. The silicone elastomer is cured and then the textured polymer film is removed, leaving a desired texture, which mirrors the texture on the polymer film, on the surface of the cured silicone elastomer. In one embodiment, the cured silicone elastomer has the shape of a breast for use as a prosthesis.

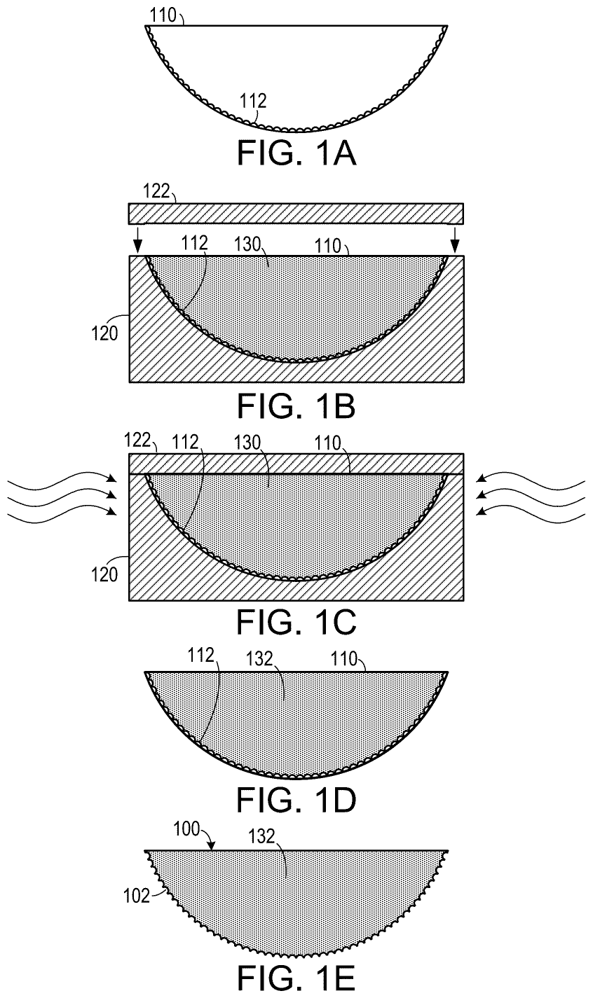

As shown in FIG. 1A, in one representative embodiment of a method of making a breast prosthesis having a textured surface, a polymer film bag 110 has an interior surface that includes a texture 112 that is complementary to the desired texture. As shown in FIG. 1B, the bag 110 is placed in a mold 120 that defines a cavity that is complementary to the shape of the breast prosthesis. An uncured silicone elastomer 130 is injected into the bag 110 (e.g, through an injection inlet, not shown) and a top 122 can be placed on the mold 120. As shown in FIG. 1C, the mold 120 and the elastomer 130 are subjected to a curing process. The curing process can include heating the mold 120. Alternatively, curing can be accomplished through the use of a catalyst added to the elastomer 130 at a predetermined time prior to placing it in the mold 120.

Once cured, the bag 110 and the now-cured elastomer 132 is removed from the mold, as shown in FIG. 1D and the polymer film bag 110 is removed from the from cured elastomer 132, leaving the breast prosthesis 100 with the desired texture 102 on at least one surface thereof.

In the invention, a polymeric film (which can be either soft and flexible or rigid) with a texture is used when casting or molding silicone elastomer to create the desired texture on the cured silicone elastomer surface. The film can be formed into a bag made by heat sealing layers of film together with an injection inlet. If transparent film is used, it is possible to remove any air bubbles after the bag has been filled. With traditional molds, the solid pieces are clamped together, and the mold interior is filled with liquid polymer. The mold must have vents for air to escape, but depending on the mold design and polymer flow, it is possible to trap air in the mold, creating defects in the molded part. Some molds are not made from transparent materials. For molds made with transparent materials, the air bubbles may be difficult to see in thick, molded parts. Also, it can be difficult to get air bubbles to the vent holes. The flexible transparent film bags used in the present invention allow air bubble to be pushed towards an inlet or vent easily. The film used can be flat and much lighter weight compared to a traditional mold, making it easier to handle. The film can also be formed to the shape of the mold with heat, pressure, a vacuum or a combination thereof.

A variety of textured, matte, or ‘soft touch’ polymeric films can be used. They can made from different polymers (including: polyethylene, polypropylene, thermoplastic polyurethane). The films obtain their texture by being laminated with a textured material, embossment, having a textured coating applied, or are cast onto a textured surface. These films are manufactured in bulk and can be inexpensive. Silicone elastomer has low adhesion to most polymer films, therefore removing the film after the silicone elastomer has cured is easy to do without damaging the cured elastomer or the molded texture. Rigid films are useful for molding thin parts or sheets of silicone elastomer. The rigid film can be laid on a flat surface or other container, with minimal to no wrinkles to smooth out. The liquid uncured silicone elastomer can be poured or painted onto the rigid film and spread to the desired thickness. The portion of the silicone elastomer in contact with the rigid film will mold to the desired texture and the side exposed to the atmosphere will not have a molded texture. Flexible and stretch films can be used for organic shapes and thicker, molded parts. The films can be textured with a wide variety of appearances—matte, shiny, decorative patterns, lines, crosshatching, waves, dots, etc.

The silicone elastomer can be cured while in contact with the textured surface of the polymeric film. After the silicone elastomer has cured, the film is removed and discarded, leaving a textured surface on the silicone elastomer. The advantage here is that a new clean piece of textured polymeric film is used each time. There is no need to maintain, resurface or rigorously clean a textured mold surface. Additionally, the polymeric film acts as a mold release, so no mold release agents are needed. The molding can be done at ambient temperature, but adding heat can accelerate the molding and curing process.

The textured polymeric film can also be sealed into a bag with an inlet and the textured surface(s) are on the interior of the bag. The bag is placed into a mold and the uncured silicone elastomer is injected into the bag. Alternatively, the bag may be filled and then placed into the mold. Then, the mold is closed while the silicone elastomer cures. The mold can be cured under pressure or with vacuum (depending on the specific elastomer or process used), to assist in the film and uncured silicone elastomer in taking the shape of the mold. Heat can also be used to aid in molding the film and silicone, as well as speeding up the cure time.

After the curing process is complete, the bag filled with cured silicone elastomer is removed from the mold. Since the silicone elastomer is encapsulated within the polymeric film bag during the curing process, there is no chance for dust, dirt, or lint to get into the uncured silicone elastomer. The bag is subsequently removed to expose the silicone elastomer with the textured surface. Alternatively, the bag can be left on as a protective layer until the molded part is ready for use or for further processing.

In one embodiment, the method is used to make a silicone elastomer breast prosthesis with a textured surface. A bag is made from soft, flexible polymeric film. This film can be a thermoplastic polyurethane film (or other type of thermoplastic) with a fine, matte microtexture. (In certain embodiments, the thermoplastic film can have a decorative texture, a microtexture, a shiny texture, a matte texture, and combinations these textures.) The surface texture is in the bag interior. The bag is sealed with the footprint of the breast shape and an injection inlet. Then the bag is placed into a mold with the corresponding breast footprint and shape. The shape can be the entire breast or a partial shell shape. Liquid silicone elastomer is injected into the bag and any air pockets are removed. The mold is closed and the liquid silicone elastomer cures while in contact with the textured bag surface. Elevated pressure or a vacuum may be applied to the breast prosthesis mold to aid in molding the film and curing the silicone elastomer. Heat can also be used to aid in molding the film and silicone, as well as speeding up the cure time. After the silicone elastomer has cured, the silicone encapsulated within the bag is removed from the mold. The bag is removed from the cured silicone, exposing the cured elastomer with a texture that is complementary to the texture on the surface of the film.

The method can be used any time a textured surface is desired on an elastomer. For example, a flat sheet of polymeric film with a textured surface can be used when a flat elastomer with a textured surface is desirable.

Although specific advantages have been enumerated above, various embodiments may include some, none, or all of the enumerated advantages. Other technical advantages may become readily apparent to one of ordinary skill in the art after review of the following FIGURES and description. It is understood that, although exemplary embodiments are illustrated in the figures and described herein, the principles of the present disclosure may be implemented using any number of techniques, whether currently known or not. Modifications, additions, or omissions may be made to the systems, apparatuses, and methods described herein without departing from the scope of the invention. The components of the systems and apparatuses may be integrated or separated. The operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set. It is intended that the claims and claim elements recited below do not invoke 35 U.S.C. § 112(f) unless the words “means for” or “step for” are explicitly used in the particular claim. The above-described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

What is claimed is:

1. A method of applying a desired texture to a surface of a silicone elastomer, comprising the steps of:
(a) applying uncured silicone elastomer to at least one side of a polymeric film in which the at least one side has a complementary texture that is complementary in shape to the desired texture;
(b) curing the uncured silicone elastomer while it is in contact with the at least one side of the polymeric film so as to form a cured silicone elastomer; and
(c) removing the polymeric film from the cured silicone elastomer so as to expose the desired texture on the surface thereof.

2. The method of claim 1, wherein the applying step comprises pouring the uncured silicone elastomer onto the at least one side of the polymeric film.

3. The method of claim 1, wherein the applying step comprises painting the uncured silicone elastomer onto the at least one side of the polymeric film.

4. The method of claim 1, wherein the polymeric film is formed into a polymeric film bag in which the at least one side forms an interior side of the polymeric film bag and wherein the applying step comprises injecting the uncured silicone elastomer into the polymeric film bag.

5. The method of claim 4, wherein the bag has an organic shape.

6. The method of claim 4, further comprising the step of placing the bag into a mold prior to the curing step.

7. The method of claim 4, wherein the polymeric film comprises a thermoplastic.

8. The method of claim 4, wherein the polymeric film comprises a thermoplastic polyurethane film.

9. The method of claim 1, wherein the polymeric film comprises a rigid polymeric film.

10. The method of claim 1, wherein the polymeric film comprises a soft and flexible polymeric film.

11. The method of claim 1, wherein the desired texture includes a texture selected from a list of textures consisting of: a decorative texture, a microtexture, a shiny texture, a matte texture, and combinations thereof.

12. The method of claim 1, wherein the curing step comprises the step of applying at least one selected one of: an elevated pressure; a vacuum; or heat to the uncured silicone elastomer.

13. A method of making a silicone elastomer breast prosthesis, comprising the steps of:
(a) placing a bag that includes a flexible polymeric film into a mold that is complementary in shape to the breast prosthesis, the polymeric bag including at least one interior surface that has a complementary texture that is complementary to a desired texture;
(b) injecting an uncured silicone elastomer into the bag;
(c) curing the uncured silicone elastomer while the uncured silicone elastomer is in the bag so as to form a cured silicone elastomer shape; and
(d) removing the bag from the silicone elastomer shape so as to expose the breast prosthesis, wherein the breast prosthesis includes at least one surface having the desired texture.

14. The method of claim 13, wherein the desired texture includes a texture selected from a list of textures consisting of: a decorative texture, a microtexture, a shiny texture, a matte texture, and combinations thereof.

15. The method of claim 13, wherein the curing step comprises the step of applying at least one selected one of: an elevated pressure, a vacuum or heat to the uncured silicone elastomer.

16. The method of claim 13, wherein the breast prosthesis has a shell shape.

17. The method of claim 13, wherein the injecting step occurs before the step of placing the bag into the mold.
